(19) United States
(12) Patent Application Publication
(10) Pub. No.: US 2006/0276716 A1
(43) Pub. Date: Dec. 7, 2006

(54) Atrial fibrillation detection method and apparatus

(76) Inventors: Jennifer Healey, Waltham, MA (US); Beth T. Logan, Cambridge, MA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/146,745

(22) Filed: Jun. 7, 2005

(51) Int. Cl. A61B 5/04

(52) U.S. Cl. 600/516; 600/518

(57) ABSTRACT

A method for automatically detecting atrial fibrillation in a non-standard ECG signal having changing morphology and containing significant muscle noise generated by an ambulatory subject is provided. A morphology independent QRS detector is used to compute R-R intervals in the ECG signal. The variance of the R-R intervals over a sliding window is normalized and compared with a threshold to determine if atrial fibrillation is present within the window.

### BACKGROUND OF THE INVENTION

As shown in FIG. 1, the heart 100 is a pump, has four chambers and is divided into a right side and a left side by a muscular wall called the septum 102. The two chambers at the top are called the right atrium 104 and the left atrium 106 and the two chambers at the bottom are called the right ventricle 108 and the left ventricle 110. The atria and ventricles work together, contracting and relaxing to pump blood out of the heart.

Oxygen-poor blood enters the top of the heart through the inferior and superior vena cava veins and flows into the right atrium 104 and passes through the tricuspid valve to the right ventricle 108. After the right ventricle 104 fills, it contracts and blood flows through the pulmonary valve to the lungs. Oxygen-rich blood from the lungs enters the left atrium 106 through the pulmonary vein and through the left atrium 106 to the left ventricle 110. The left ventricle pumps the blood into the aorta.

The heart also has an electrical system that includes a pacemaker to control the contraction of the heart chambers. Normal heart rhythm is termed sinus rhythm. During normal sinus rhythm, the heartbeat starts with a miniature electrical impulse in the sinoatrial (SA) node 116, also referred to as the heart's “natural pacemaker” located in the right atrium 104. The electrical signal spreads across the atria and via the atrioventricular (AV) node 112 to the ventricles. The AV node 112 creates a brief delay (about one tenth of a second) in the impulse to allow the atria to contract and force blood into the ventricles and then spreads rapidly across the ventricles to make them contract. The AV node 112 connects to a group of fibers (the His-Purkinje system) 114 in the ventricles that conducts the electrical signal. The ventricles are the muscular part of the heart that actually pump the blood. The ventricles are electrically isolated from the atria and electrical signals reach them via the AV node 112.

An electrocardiogram (ECG or EKG) is a graphic tracing of the variations in electric potential caused by the excitation of the heart muscle plotted along a time axis. The variations in electric potential are detected at the body surface through electrodes that are placed on different parts of the body (limbs, chest wall). The signals are amplified and recorded by the electrocardiograph. The electrocardiograph is an instrument for recording the changes of electrical potential. The ECG records the depolarization (stimulation) and repolarization (recovery) potentials generated by the atrial and ventricular myocardium.

FIG. 2 is a schematic illustration of an output from an electrocardiogram for a normal heart rhythm. The electrocardiogram shows the deflections resulting from atrial and ventricular activity. A typical electrocardiogram consists of a regular sequence of deflections (waves), labeled P, QRS, T and U. The first deflection (P) is due to excitation (contraction) of the atria. The QRS deflections are due to excitation (depolarization) of the ventricles. The T wave is due to recovery of the ventricles (repolarization). The U wave is a potential undulation of unknown origin immediately following the T wave. The amplitude of each of these components (deflections) is dependent on the orientation of the heart within the individual and the electrodes used to record the ECG.

The heart rate is the number of times the heart beats per minute which can be calculated by counting the average number of beats for a given duration (typically 15-30 seconds). The linear distance between neighboring peaks of simultaneous heart beats on an ECG corresponds to the time necessary for a single cardiac cycle (heart beat). As illustrated in FIG. 2, the linear distance (labeled “time”) is measured between the peaks of neighboring QRS complexes.

The distance between the R waves in a given ECG signal is variable. When an ECG is performed, it is common to measure the heart rate for several cardiac cycles to determine how consistently the heart beats. In addition to analyzing whether the interval between waves from consecutive cardiac cycles remain consistent, the individual that analyzes the ECG also looks for how fast the heart is beating, the consistent shape of each wave, and the normality of duration and configuration of each wave.

An arrhythmia is a change in rhythm of the heartbeat. Atrial fibrillation (AF) is a common sustained arrhythmia in which the atria contract rapidly and irregularly in a chaotic manner due to multiple electrical signals firing at 400 to 600 beats per minute. The AV node 112 (FIG. 1) filters out most of the additional electrical signals. However, more electrical signals reach the ventricles 108, 110 (FIG. 1) than normal, resulting in the ventricles beating at rates of 110 to 180 beats per minute faster than normal resting heart rate which is between 60 and 80 beats per minute.

AF is not immediately life threatening, but the risk of stroke is increased because the quivering atria beat too rapidly to contract effectively and with time they enlarge, which can lead to blood clots forming within the atria. If a blood clot leaves the heart and lodges in the brain, a stroke results. Also, the rapid beating of the ventricles for prolonged periods can result in weakening them which can lead to heart failure.

Often the symptoms of atrial fibrillation occur infrequently and can only be detected by continuous monitoring over a long time period on an ambulatory subject using a small portable ECG recorder, called a Holter monitor (continuous ambulatory electrocardiograph monitor). Electrodes are taped to the chest and wires are connected to a portable battery-operated recorder.

The ECG signal generated by the ambulatory monitor often contains significant muscle noise as the subject is ambulatory which makes it difficult to detect AF. Furthermore, due to the limitation in available memory in the ambulatory monitor, only a fixed length recording can be stored, so the individual may be asked to only record when recognizing a rapid heart beat or the device may record over previously recorded data.

In addition, standard practice for using the ambulatory monitor requires electrode placement in known positions on the body in order to perform either visual analysis or analysis by a computer program using template matches on the recorded signal because the QRS complex differs dependent on position of the electrode. The analysis of each trace is dependent on the position of the electrode corresponding to the trace. The analysis involves comparing the trace with a stored template of a normal trace at the same position. Thus, the placement of the electrodes is critical to the analysis and is performed by a person who has received special training in the placement of the electrodes. The need for a person skilled in placement of the electrodes increases the cost of the ECG and limits the use of the test to those who have already exhibited symptoms.

Furthermore, the electrodes are attached to the body through the use of an adhesive so that they remain in place during the test period. Long term use, for example, for a time period greater than 24 hours, can result in skin irritation due to exposure to the adhesive.

### SUMMARY OF THE INVENTION

The present invention allows the electrodes to be placed in any position on the chest wall, that is, it allows changing morphology by allowing positioning of an electrode in a non-standard location.

In particular, the present invention provides a solution for detecting atrial fibrillation in non-standard lead configurations, in a noisy signal from an ambulatory subject from a sensor rotated through multiple placements. Atrial fibrillation can be detected from a low cost sensor which may be a small form factor sensor with one inch lead separation.

The nature of the detection technique (method and apparatus), in terms of robustness and ECG sensor device morphology independence, allows it to be used in non-standard electrode lead placements and allows seamless detection as the electrode (sensor) is moved to different positions. This enables the sensor device (electrode) to be placed in a different location each day, which reduces skin irritation in any one particular location, allowing long term wearability. The robustness of the algorithm to non-standard electrode lead placements does not require placement by someone who has received special training in the placement of the electrodes which decreases the cost of the testing and allows the device to be used for preventative care. As the analysis is morphology independent, it does not require a match with a template for a normal trace for a particular position.

In a preferred embodiment, a computer implemented method for automatically detecting atrial fibrillation in an ECG signal detects QRS complexes in the ECG signal. Based on an interval between successive peaks in the detected QRS complexes, a variance of normalized intervals over a sliding window in the ECG signal is computed and the variance is compared with a threshold to provide an indication of whether atrial fibrillation is present in the window.

An indication of whether atrial fibrillation is present in a beat window in the ECG signal may be provided dependent on a number of windows within the beat window in which atrial fibrillation has been detected. The analysis of the ECG signal may be performed in a server remote from a sensor that captures the ECG signal by continuous monitoring of cardiac activity on an ambulatory subject.

The detection of QRS complexes in the ECG signal may be morphology independent, that is, independent of a position of a sensing electrode. In one embodiment, the window is 10 seconds, the beat window is 600 beats and the settable threshold is 200.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a diagram of a heart;

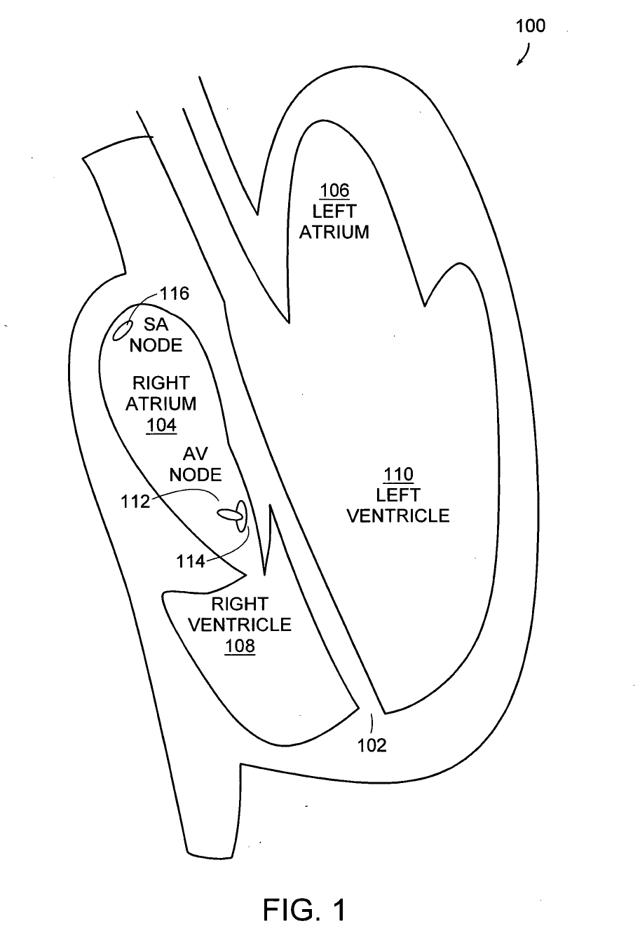

FIG. 2 is a schematic illustration of an output from an electrocardiogram for a normal heart rhythm;

FIG. 3 is a block diagram of typical computer system in which the present invention is utilized;

FIG. 4 is a flow diagram of a preferred embodiment of the method for detecting AF according to the principles of the present invention;

FIGS. 5A-5F are graphs of the line transform on three ECG morphologies, upward, downward and cross;

FIG. 6 is a histogram of R-R interval variance calculated over a 10 second window for AF data;

FIG. 7 is a histogram of R-R interval variance calculated over a 10 second window for normal data;

FIG. 8 is a schematic illustration of patch and electrode placement on the individual;

FIG. 9 is a graphical representation of errors for the QRS detector for each pair of electrodes shown in FIG. 8;

FIG. 10 is a graph illustrating results for different embodiments of the method shown in FIG. 4; and

FIG. 11 is an example of a summary of the analysis of the ECG testing output from the AF detector that is displayed on a screen of a computer monitor.

### DETAILED DESCRIPTION OF THE INVENTION

A description of preferred embodiments of the invention follows.

Most ECG recordings contain two or more simultaneously recorded ECG signals, called “leads.” The heart generates an electrical field that varies spatially as well as temporally. Thus, the standard practice is to record two or more signals (leads) derived using sensing electrodes placed at certain specific locations. The wires that connect the electrodes to the recording equipment are also sometimes referred to as “leads”.

As is well-known in the art, there is a standard placement for ECG leads that requires an individual with special training to perform the placement. Typically, a nurse performs the placement, a doctor performs the testing, an ECG technician runs the analysis software and a cardiologist performs the over-read. Non-ambulatory electrocardiograph devices include precordial leads and limb leads. Precordial leads are placed on the chest wall at pre-defined positions on the chest wall referred to as V1-V6. Position V1 is in the fourth intercostal space at the right sternal border, V2 is in the fourth intercostal space at the left sternal border, V3 is mid-way between V2 and V4, V4 is in the fifth intercostal space in the mid-clavicular line, V5 is in the left anterior axillary line at the level of V4 and V6 is in the left mid-axillary line at the level of V4. The limb leads are placed on the right and left wrists and the right and left ankles. Limb leads are not generally used in ambulatory electrocardiograph devices because physical activity causes significant interference in these leads.

A major problem with ECG is the difficulty of ensuring that electrodes are properly positioned. The present approach addresses the problem of detecting the heart condition known as atrial fibrillation (AF) using an ambulatory electrocardiograph device that does not require placement of electrodes at specified positions on the body

In one embodiment, an ambulatory electrocardiograph device is designed for long term (greater than 24 hours) wearability and as such is small with electrodes much closer together than is typical in clinical applications. Sensors attachable to the chest wall can be moved daily to a randomly chosen position on the chest wall by the individual being tested to avoid skin irritation from the electrode adhesives. The electrocardiogram (ECG or EKG) signal generated by the ambulatory electrocardiograph device is non-standard, has changing morphology (form and structure) and contains significant muscle noise because the individual is ambulatory while being monitored.

The ambulatory electrocardiograph device can include memory for storing the ECG signal received through the electrode. The ambulatory electrocardiograph can periodically download the stored ECG signal to a computer system for analysis. In one embodiment, the stored ECG signal is downloaded to the computer system through a wireless communication interface to a wireless network. In an alternative embodiment, the stored ECG signal is transmitted to the computer system over a telephone network. In yet another embodiment, the ECG signal is stored in removable storage in the ambulatory device for later analysis by another computer system.

In one embodiment, the computer system that analyzes the ECG signal is separate from the ambulatory electrocardiograph device (sensor) in order to conserve power on the device, and to reduce the cost and weight of the device by using remote computations and storage of the recorded ECG signal. The ECG signal is analyzed at a remote site from the sensor by a technician or other qualified person and the individual receives notification of the verified results. In order to prevent the individual being monitored from receiving notification of false triggers, notification of detection of AF is hidden from the individual by analyzing the ECG signal at a remote site. In an alternate embodiment, the analysis can be performed in the sensor with the notification of AF being hidden from the individual being monitored, for example, the notification can be transmitted to a remote computer system without alerting the individual.

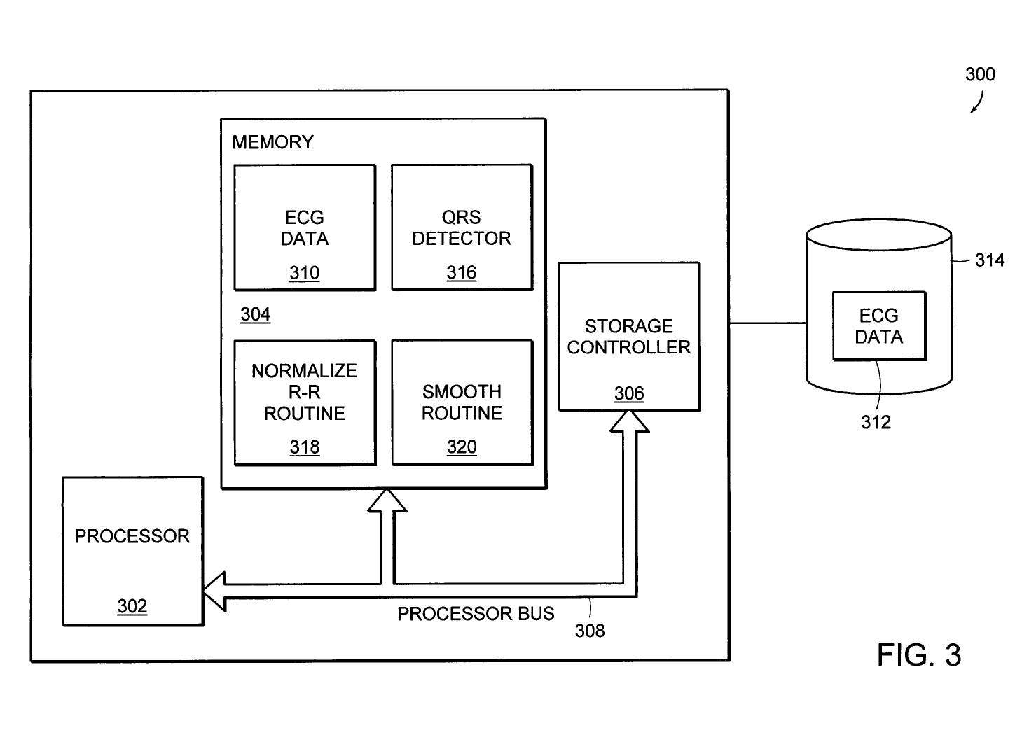

FIG. 3 is a block diagram of a computer system 300 in which the present invention is utilized. The computer system includes a processor 302, memory 304 and a storage controller 306 coupled to secondary memory such as, a disk drive 314. The processor 302 is coupled to the memory 304 and the storage controller 306 through a processor bus 308. The storage controller 306 controls the disk drive 314.

The ECG signals (ECG data) collected by ECG devices such as an ambulatory device can be stored in ECG data 310 in the memory 304 or in ECG data 312 on the disk drive 314. The memory 304 also stores routines executable by the processor 302 to automatically detect atrial fibrillation in the ECG signal. The routines include a QRS detector 316 that computes R-R intervals in the ECG data. A normalize R-R routine 318 normalizes the R-R intervals computed by the QRS detector 316 and uses the computed R-R intervals to compute a statistic over a sliding window to detect AF. In one embodiment, the sliding window is 10 seconds. A smoothing routine 320 eliminates spurious errors due to noise in the normalized R-R intervals.

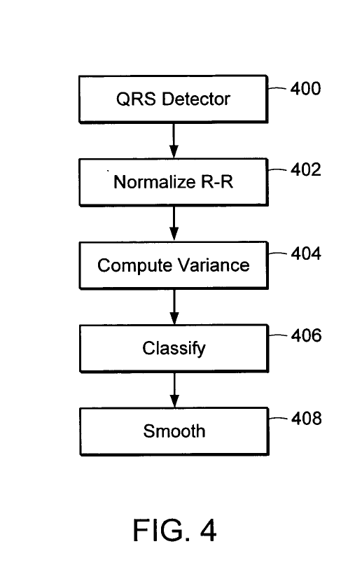

FIG. 4 is a flowchart illustrating a technique for detecting AF in the ECG signal according to the principles of the present invention.

At step 400, a morphology-independent single-channel QRS detector routine 316 is used to determine R-R intervals in the ECG signal (lead) by detecting the QRS complex.

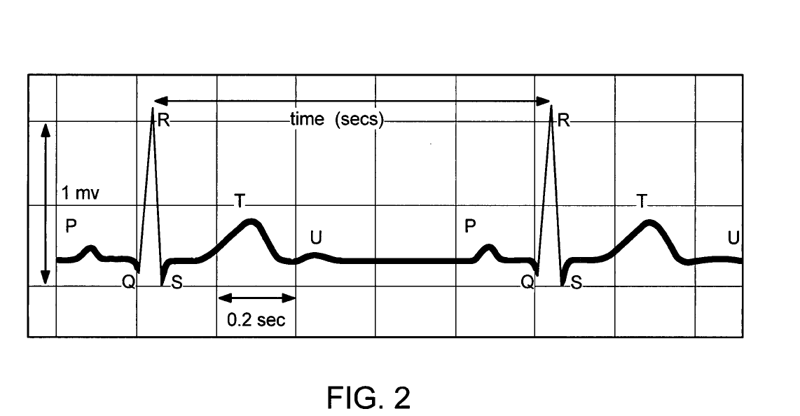

ECG leads record the difference in potential between electrodes placed on the surface of the body. Returning to FIG. 2, the ECG waves (deflections) are labeled alphabetically starting with the P wave. The P wave represents atrial depolarization. The QRS complex represents ventricular depolarization. The ST-T-U complex (ST segment, T wave, and U wave) represents ventricular repolorarization. There are four major ECG intervals: R-R, PR, QRS and QT.

The QRS complex is subdivided into specific deflections or waves. If the initial QRS deflection in a given ECG lead is negative, it is termed a Q wave. The first positive deflection is termed an R wave. A negative deflection after an R wave is an S wave.

The heart rate can be computed from the R-R interval. AF is characterized by disorganized atrial activity, resulting in an ECG without discrete P waves. A secondary effect of this disorganized atrial activity is irratic ventricular contraction, resulting in an ECG with high R-R interval variation. The variance of R-R intervals in an ECG signal is a good indicator of atrial fibrillation. If there is an indication of atrial fibrillation further testing can be performed on the subject. Although this test is not as accurate as other available tests, it is inexpensive to perform and provides an indication as to whether more expensive testing is warranted.

The shape of the QRS complex in an electrocardiogram differs depending on where the sensing electrodes are placed on the body. The R-wave spike in the QRS complex can either be upwards pointing, downwards pointing or can have both up and down components. In standard 12-lead electrocardiography, six successively placed leads along the first floating rib from the midline of the subject body to the side will normally show an “R-wave progression” from an upwards to a downwards spike. A morphology independent QRS detector allows accurate calculation of R-R intervals regardless of the sensor's placement on the chest wall. One such morphology independent QRS detector is described in “A Robust Open-source Algorithm to Detect Onset and Duration of QRS Complexes”, W. Zong, G. B. Moody, and D. Jiang, Computers in Cardiology 2003, 30:737-740, the contents of which are incorporated herein by reference in its entirety. This QRS detector routine is an open-source routine and is available at www.physionet.org/physiotools/wag/wqrs-1.htm. This technique implements a linear transform of the ECG signal. Using this transform, for each time window, w, the length of the line of the ECG signal over that time is calculated. The result is a line transform of the ECG where each point represents a successive line integral of a sliding window, w. The QRS spike is the most prominent feature in an ECG signal and the least affected by muscle noise allowing the R-R intervals to be computed.

In one embodiment, the QRS detector 316 uses the WQRS single-channel QRS detector described in W. Zong, G. B. Moody, D. Jiang, “A Robust Open-source Algorithm to Detect Onset and Duration of QRS Complexes” Computers in Cardiology 2003, 30:737-740. The QRS detector 316 detects onset of QRS complexes and is insensitive to QRS morphology change. A non-linear scaling factor for ECG curve length enhances the QRS complex and suppresses other parts of the ECG signal and noise.

The QRS detector routine 316 detects onset and duration of QRS complexes. Using the QRS detector routine, the ECG signal (data) 310 stored in memory 304 in the AF detection system is input to a low-pass filter which produces a filtered ECG signal. The ECG data can be a low quality ECG signal that has been received from an ambulatory electrocardiogram device. For the adult human, the ideal passband for the low-pass filter is about 5-15 Hz. The filtered ECG is input to a curve length transformation which converts the filtered ECG signal to a curve length signal. The QRS detector 316 is stable and insensitive to QRS morphology change. The curve length transformation converts the filtered ECG signal to a curve length signal by introducing a nonlinear scaling factor to enhance the QRS complex and suppress unwanted noise.

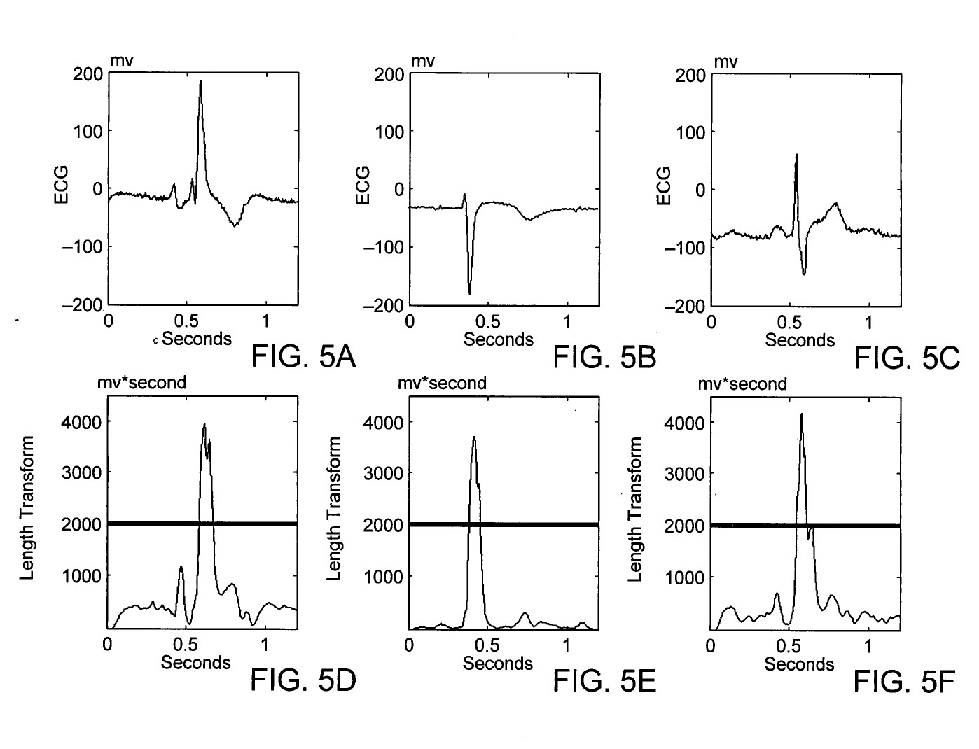

FIGS. 5A-5F illustrate the effect of the curve length (line) transform on three ECG morphologies: upward (FIG. 5A), downward (FIG. 5B) and cross (FIG. 5C). The tip of the QRS spike is about 200 mV after being amplified by the sensor in FIG. 5A, at about −200 mV in FIG. 5B and about 50 mV in FIG. 5C. FIG. 5D is a line transform corresponding to the upward ECG morphology shown in FIG. 5A. FIG. 5E is a line transform corresponding to the downward ECG morphology shown in FIG. 5B. FIG. 5F is a line transform corresponding to the cross ECG morphology shown in FIG. 5C. Referring to FIGS. 5A-5F, regardless of morphology, each corresponding line transform (FIGS. 5D-5F) peaks at the QRS complex at about 400 mV*second allowing the QRS spike to be found. The R-R interval is used to determine the arithmetic mean of an R-R interval sequence.

Returning to FIG. 4, at step 402, each R-R interval is normalized in order to normalize across individuals with different resting heart rates. First, the arithmetic mean for an R-R interval (i) is computed using the following equation
RRmean(i)=0.75*RRmean(i−1)+0.25*RR(i)

where:
RR(i) is the current RR interval; and
RRmean(i−1) is the mean computed for the prior RR interval.

The computation of the arithmetic mean for an R-R interval is discussed for use in feature normalization in Moody, George and Mark Roger, “A New Method for Detecting Atrial Fibrillation Using R-R Intervals,” Computers in Cardiology 1983 incorporated herein by reference.

After the arithmetic mean has been computed, the R-R interval is normalized using the following equation:
RRnorm=RR/RRmean*100

The R-R interval is normalized because it is dependent on the resting heart rate which differs between individuals. By normalizing the R-R interval, an AF threshold can be selected that is independent of the individual being monitored.

At step 404, the variance of the RRnorm statistic is computed over 10 second sliding windows. The selection of a 10 second sliding window is a tradeoff between having enough beats to obtain a good estimate of the R-R interval variance and having a small enough window so that the majority (preferably all) of the heart beats within the window are either AF or non-AF. The window is sliding to eliminate edge effects and to allow the normalize R-R routine to operate on streaming data.

The variance is a measure of how spread out a distribution is. The variance (var) is computed as the average squared deviation of each R-R Interval from the mean R-R interval using the following equation:
var=(sumsq)/(N−1)−N/(N−1)*(sum/N)*(sum/N)

where: sumsq=sum of the square of the normalized R-R intervals over the 10 s window;
sum=sum of the normalized R-R intervals over the 10 second window;
N=number of R-R intervals in the 10 second window.

As the window slides, the latest beat is added and the oldest beats are dropped until the window size is less than or equal to 10 seconds. The sum and sumq are adjusted to account for beats added and dropped and the variance is computed.

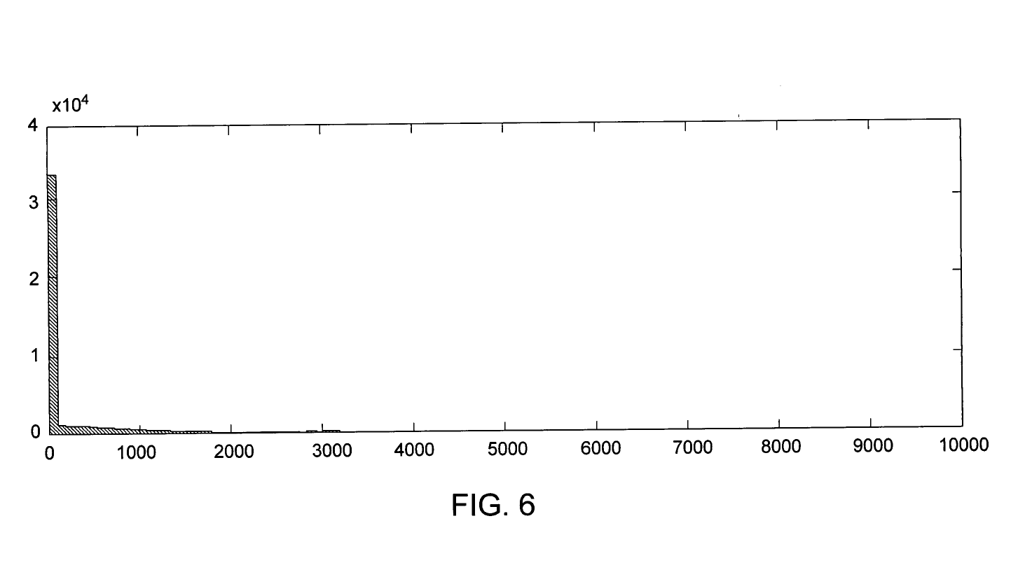

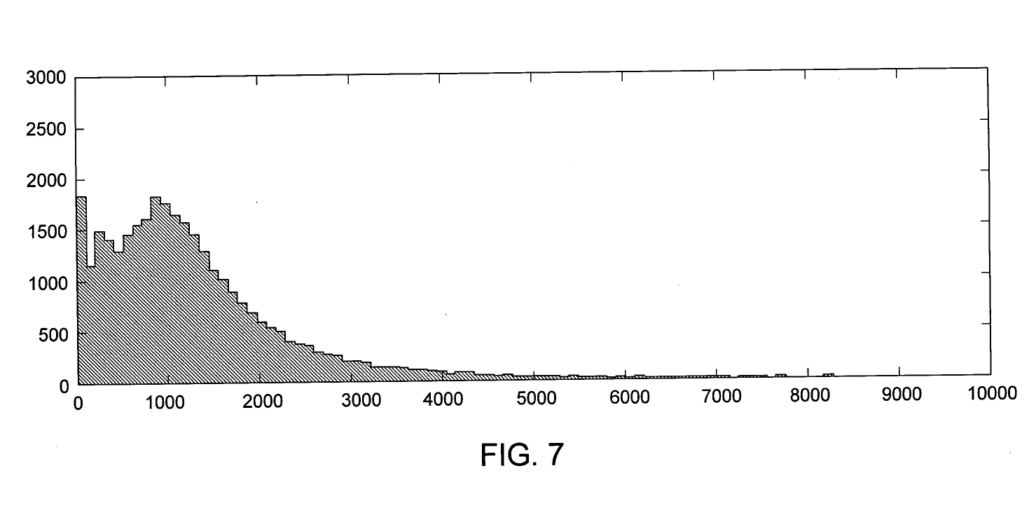

FIGS. 6 and 7 are histograms of variance of R-R intervals calculated over 10 second windows. The histograms illustrate variance versus the number of windows having that variance value. These histograms were computed for heart data from the MIT-BIH AF database available at www.physionet.org/physiobank/database/afdb/. FIG. 6 is a histogram for normal heart data and FIG. 7 is a histogram for data identified as AF. Normal heart data and AF data exhibit different characteristics which can be used to classify ECG signals as normal or AF. As shown, the variance decreases as the heart rate increases so the heart rate is normalized. The variable variance shown in FIG. 7 for AF data is an indication of AF. For a normal ECG signal, the variance is less than 1000 with the majority of windows (3.3×10⁴ having a variance that ranges from 0 to about 200). In the example shown for an ECG signal with AF, the variance ranges from 0 to 6000 with the majority of windows having a variance greater than 200. In the example shown, less than 2000 windows have a variance between 0 and 200.

Returning to FIG. 4, at step 406, the computed variance over each 10 second window is compared with a settable threshold in order to compute an initial AF detection. In one embodiment, the settable threshold is 200 because the variance range for a normal ECG is below 200 as shown in FIG. 6.

At step 408, further processing can be performed to eliminate spurious errors. For example, a smoothing algorithm such as, a simple majority voting scheme over a number of beats can be used or any other smoothing algorithm well known to those skilled in the art. In one embodiment, the smoothing algorithm counts how many times the AF was detected in each 10 second window in step 406 over 600 beats, that is, a time period of about 10 minutes for a normal heart rate of 60 beats per minute. The 600 beat window was selected to set the sensitivity threshold for AF detection so that the AF detection is not too sensitive, that is, short (less than 10 minute) episodes of AF are not detected.

The 600 beat window is classified as having AF dependent on the number of 10 second windows having AF. In one embodiment, the 600 beat window is classified as having AF if 301 of the 10 second sliding windows have AF, that is, a simple majority.

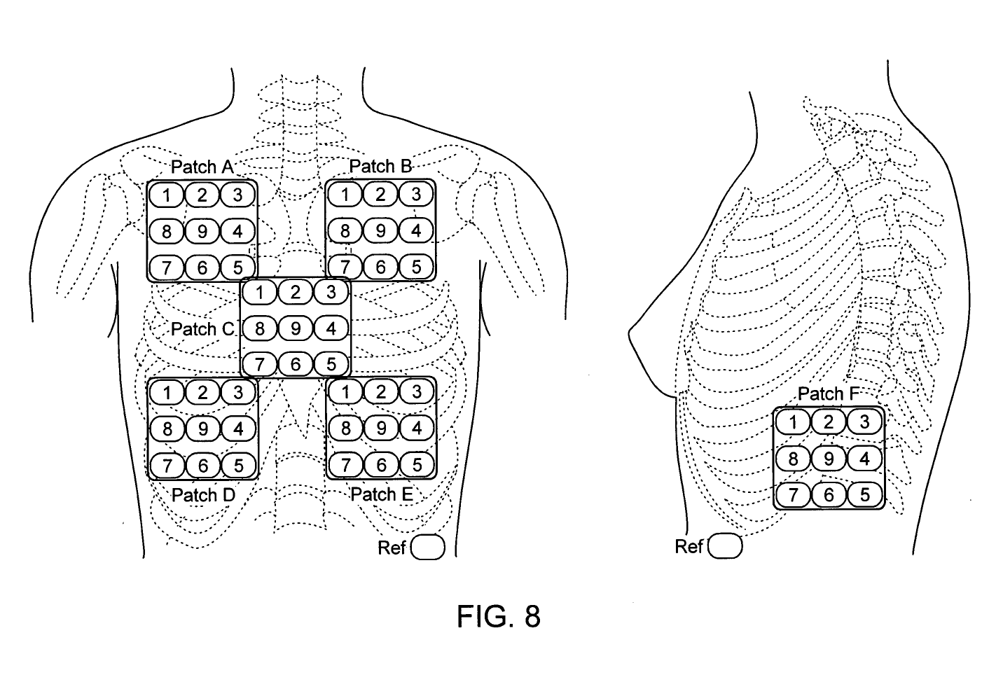

The accuracy of the morphology-independent QRS detector 316 described above was tested by performing the following experiment. ECG signals were collected from 16 individuals for conditions such as supine, sitting, standing and walking. For each individual, ECG signals were simultaneously collected from 54 electrodes. The electrodes were applied to the chest wall of the individual using six patches (labeled A-F). In the embodiment shown, each patch included nine electrodes. FIG. 8 illustrates placement of patches on the chest wall. The signals formed by arithmetically subtracting pairs of electrodes were studied in order to simulate the likely ECG signal from the proposed sensor in different positions.

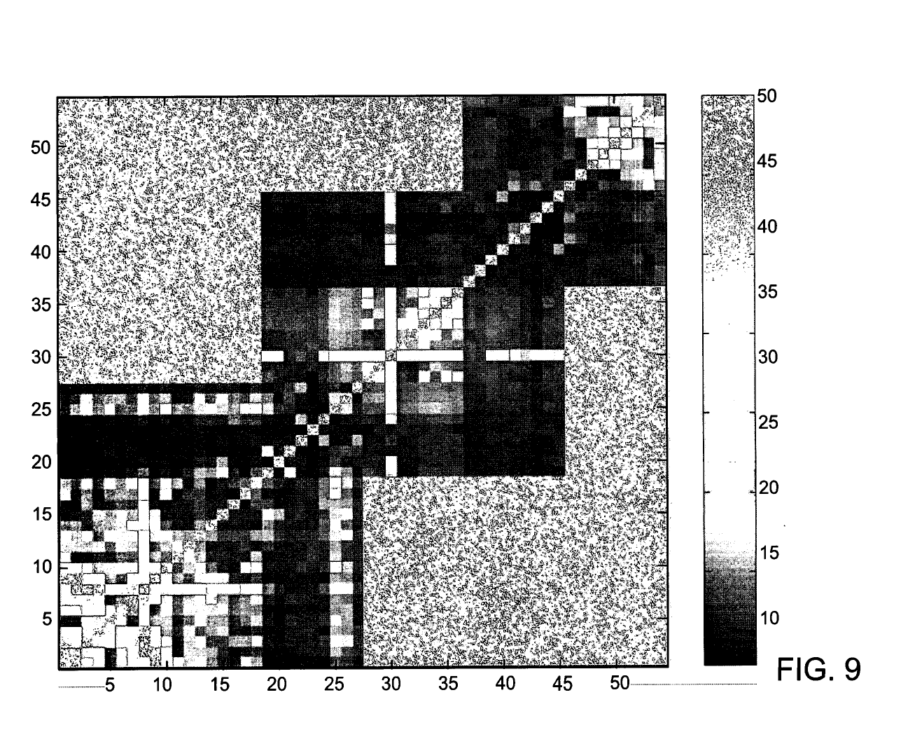

FIG. 9 is a graphical representation of the number of detection errors for each differential pair of electrodes shown in FIG. 8 for the QRS detector averaged over all individuals and conditions. For example, location (1, 3) on the graph represents the error in the differential signal between electrode 1 and electrode 3 on Patch A. For the purpose of this graph, the numbers 1-9 represent electrodes 1-9 on Patch A, the numbers 10-18 represent electrodes 1-9 on Patch B, the numbers 19-27 represent the electrodes 1-9 on Patch C, the numbers 28-36 represent the electrodes 1-9 on Patch D, the numbers 37-45 represent the electrodes 1-9 on Patch E, and the numbers 46-54 represent the electrodes 1-9 on Patch F. Only pairs of electrodes on the same or adjacent patches were considered. The areas on the graph corresponding to an error in the range 40-50, other than the areas corresponding to electrode 2 on Patch A and electrode 2 on Patch B were not considered. For example, electrodes on Patches D and E were not paired with electrodes on Patches A and B because the patches are not adjacent.

The error was computed by scoring the QRS annotations from each differential pair against a reference lead (FIG. 5, 800) using the ANSI/AAMI standard beat-by-beat annotation file comparator bxb (see www.physionet.org/physiotools/wfdb/bxb.c).

The bxb program implements the beat-by-beat comparison algorithms described in AAMI/ANSI EC38:1998, the American National Standard for ambulatory ECGs, and in AAMI EC57:1998, the American National Standard for Testing and Reporting Performance Results of Cardiac Rhythm and ST Segment Measurement Algorithms. These standards are available from AAMI, 1110 N Glebe Road, Suite 220, Arlington, Va. 22201 USA (http://www.aami.org/).

Basically, bxb compares the location and type of beat provided by the QRS detector indicated with the location and type of beat provided by a reference. The reference lead for the differential pairs was provided by marking one of the leads. If the location detected by the QRS detector is “close enough” by the standards set out be the AAMI/ANSI and is of the same type as the reference, the annotation is judged to be “correct” otherwise it is incorrect. A confusion matrix of hits and misses is provided by the program as a result.

As shown in FIG. 9 the shade of grey indicates the error for the differential pair, black indicating the differential pairs having the lowest error. Thus, the line transform used to detect the QRS complex is robust to a wide variety of morphologies and muscle movements as required for long-term sensor wearability.

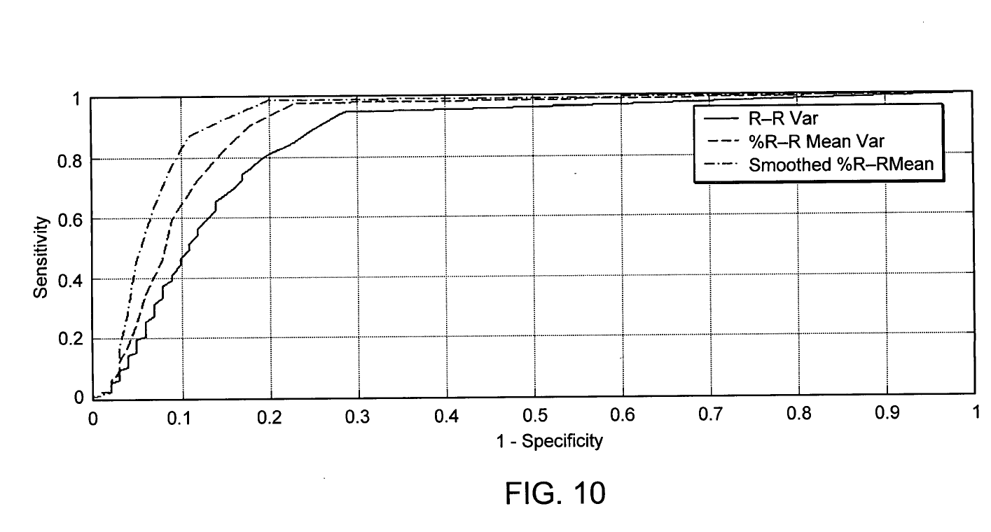

The method for detecting AF described in conjunction with FIG. 4 was tested on the MIT-BIH AF database (www.physionet.org/physiobank/database/afdb/). FIG. 10 is a graph illustrating three variants of the method described in conjunction with FIG. 4. The first, denoted “R-R Var”, is without beat normalization or smoothing (steps 402 and 408). The second, denoted “% R-R Mean Var” incorporates beat normalization, and the third “Smoothed % R-R Mean Var” employs all of the steps shown in FIG. 4. The graph illustrates that the R-R interval normalization and smoothing steps improve performance of the detection of AF in the morphology independent ECG signal.

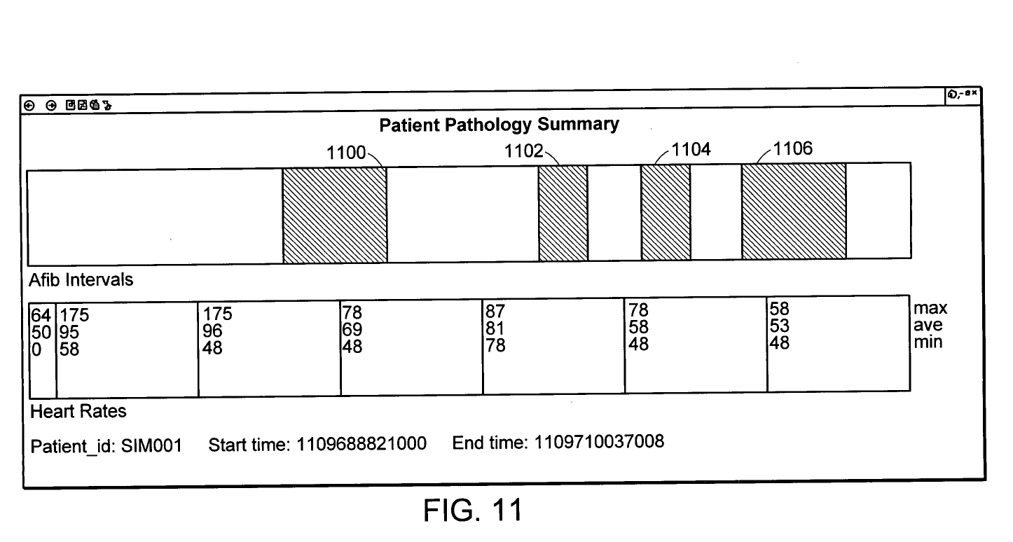

FIG. 11 is an example of a summary of the analysis of the ECG testing output from the AF detection system that is displayed on a screen of a computer monitor. The summary includes two graphs. The upper graph indicates the sections 1100, 1102, 1104, 1106 of the ECG signal in which AF was detected. The lower graph shows hourly intervals with maximum, average and minimum heart rate detected over each hour. The hour at the left of the graph is represented as being smaller than the other hours because the earlier data is “squashed” in order to display the later data. The patient identifier, and the start and end time of the test is also displayed.

It will be apparent to those of ordinary skill in the art that methods involved in the present invention may be embodied in a computer program product that includes a computer usable medium. For example, such a computer usable medium may consist of a read only memory device, such as a CD ROM disk or conventional ROM devices, or a random access memory, such as a hard drive device or a computer diskette, having a computer readable program code stored thereon.

While this invention has been particularly shown and described with references to preferred embodiments thereof, it will be understood by those skilled in the art that various changes in form and details may be made therein without departing from the scope of the invention encompassed by the appended claims.

1. A computer implemented method for automatically detecting atrial fibrillation in an ECG signal comprising:
detecting QRS complexes in the ECG signal including computing R-R invervals;
based on the computed R-R intervals in the detected QRS complexes, normalizing the R-R intervals and computing a variance of the normalized intervals over a sliding window in the ECG signal; and
comparing the computed variance with a threshold to provide an indication of whether atrial fibrillation is present in the window.

2. The method of claim 1 further comprising:
providing an indication of whether atrial fibrillation is present in a beat window in the ECG signal dependent on a number of sliding windows within the beat window in which atrial fibrillation has been detected.

3. The method of claim 1, wherein analysis of the ECG signal is performed in a server remote from a sensor that captures the ECG signal by continuous monitoring of cardiac activity on an ambulatory subject.

4. The method of claim 1, wherein the sliding window is less than or equal to 10 seconds.

5. The method of claim 1, wherein detecting QRS complexes is morphology independent.

6. The method of claim 1, wherein the threshold is 200 or greater.

7. The method of claim 2, wherein the beat window is 600 beats.

8. A computer apparatus for automatically detecting atrial fibrillation in an ECG signal comprising:
a QRS detector stored in a memory which detects QRS complexes in the ECG signal and computes R-R intervals; and
a normalize routine which normalizes the computed R-R levels and computes a variance of the normalized intervals over a sliding window in the ECG signal, the normalize routine comparing computed variance with a threshold to provide an indication of whether atrial fibrillation is present in the window.

9. The apparatus of claim 8 further comprising:
a smoothing routine which provides an indication of whether atrial fibrillation is present in a beat window in the ECG signal dependent on a number of sliding windows within the beat window in which atrial fibrillation has been detected.

10. The apparatus of claim 8, wherein analysis of the ECG signal is performed in a server remote from a sensor that captures the ECG signal by continuous monitoring of cardiac activity on an ambulatory subject.

11. The apparatus of claim 8, wherein the sliding window is less than or equal to 10 seconds.

12. The apparatus of claim 8, wherein, detecting QRS complexes is morphology independent.

13. The apparatus of claim 8, wherein the threshold is 200 or greater.

14. The apparatus of claim 9, wherein the beat window is 600 beats.

15. An apparatus for automatically detecting atrial fibrillation in an ECG signal comprising:
means for detecting QRS complexes in the ECG signal, the means for detecting computing R-R intervals;
based on the computed R-R intervals, means for normalizing the R-R intervals and for computing a variance of normalized intervals over a sliding window in the ECG signal; and
means for comparing the computed variance with a threshold to provide an indication of whether atrial fibrillation is present in the window.

16. A computer program product for automatically detecting atrial fibrillation in an ECG signal, the computer program product comprising a computer usable medium having computer readable program code thereon, including program code which:
detects QRS complexes in the ECG signal;
computes R-R intervals in the detected QRS complexes;
normalizes the computed R-R intervals;
computes a variance of normalized intervals over a sliding window in the ECG signal; and
compares the variance with a threshold to provide an indication of whether atrial fibrillation is present in the window.

17. A computer implemented method for automatically detecting atrial fibrillation in an ECG signal comprising:
detecting QRS complexes in the ECG signal;
based on an interval between successive peaks in the detected QRS complexes, computing a variance of normalized intervals over a sliding window in the ECG signal; and
comparing the variance with a threshold to provide an indication of whether atrial fibrillation is present in the window, wherein the threshold is settable and is at least one time set to 200.

18. A computer implemented method for automatically detecting atrial fibrillation in an ECG signal comprising:
detecting QRS complexes in the ECG signal;
based on an interval between successive peaks in the detected QRS complexes, computing a variance of normalized intervals over a sliding window in the ECG signal;
comparing the variance with a threshold to provide an indication of whether atrial fibrillation is present in the sliding window; and
providing an indication of whether atrial fibrillation is present in a beat window in the ECG signal dependent on a number of sliding windows within the beat window in which atrial fibrillation has been detected, wherein the beat window is about 600 beats.

19. A computer apparatus for automatically detecting atrial fibrillation in an ECG signal comprising:
a QRS detector stored in a memory which detects QRS complexes in the ECG signal; and
a normalize routine which based on an interval between successive peaks in the detected QRS complexes, computes a variance of normalized intervals over a sliding window in the ECG signal and compares the variance with a threshold to provide an indication of whether atrial fibrillation is present in the window, wherein the threshold is settable and is at least one time set to 200.

20. A computer apparatus for automatically detecting atrial fibrillation in an ECG signal comprising:
a QRS detector stored in a memory which detects QRS complexes in the ECG signal;
a normalize routine which based on an interval between successive peaks in the detected QRS complexes, computes a variance of normalized intervals over a sliding window in the ECG signal and compares the variance with a threshold to provide an indication of whether atrial fibrillation is present in the sliding window; and
a smoothing routine which provides an indication of whether atrial fibrillation is present in a beat window in the ECG signal dependent on a number of sliding windows within the beat window in which atrial fibrillation has been detected, wherein the beat window is about 600 beats.
